(19) United States
(12) Patent Application Publication
(10) Pub. No.: US 2002/0018768 A1
(43) Pub. Date: Feb. 14, 2002

(54) Control of gene expression

(75) Inventor: Batsheva Kerem, Mevaseret Zion (IL)

Correspondence Address: NATH & ASSOCIATES PLLC, Sixth Floor, 1030 15th Street, N.W., Washington, DC, 20005, US

(73) Assignee: YISSUM RESEARCH DEVELOPMENT COMPANY OF THE HEBREW UNIVERSITY OF JERUSALEM, Jerusalem (IL)

(21) Appl. No.: 09/871,809

(22) Filed: Jun. 4, 2001

Related U.S. Application Data
(63) Continuation-in-part of application No. 09/421,891, filed on Oct. 21, 1999, now abandoned.

(51) Int. Cl. A61K 48/00

(52) U.S. Cl. 424/93.21; 514/44

(57) ABSTRACT

The invention concerns a method for treating various genetic diseases caused by aberrant splicing by utilizing factors which can modulate alternative splicing. The method of the present invention is especially suitable for the treatment of cystic fibrosis.

### FIELD OF THE INVENTION

This invention relates to a method for the treatment of genetic diseases.

The following publications are believed to be relevant to the invention.

1. Augarten, A., Kerem, B. S., Yahav, Y. Noiman S., Rivlin Y., Tal, A., Blau, H., et al., Lancet, 342:25-26, (1993).

2. Biamonti, G., Buvoli, M., Bassi, M. T., Morandi, C., Cobianchi, R., Riva, S., J. Mol. Biol., 207:491-503, (1989).

3. Caceres, J. F., Stamm, S., Helfman, D. M., Krainer, A. R, Science, 265:1706-1709, (1994).

4. Cbiba-Falek, O., Kerem, E., Shoshani, T., Aviram, M., Augarten, A., Bentur, L., Tal, A., et al., Genomics, 53:276-283, (1998).

5. Chu, C. S., Trapnell, B. S., Curristin, S., Cutting, G. R., Crystal, G., Nat. Genet., 3:151-156, (1993).

6. Haramura, A., Caceres, J. F., Mayeda, A., Franza, B. R., Krainer, A. R., RNA, 4:430-444, (1998).

7. Highsmith, W. E., Burch, L. H., Zhou, Z., Olsen, J. C., Boat, T. E., Spock, A., Gorvoy, J. D., et al., N. Engl. J. Med., 331:974-980, (1994).

8. Kerem, E., Rave-Harel, N., Augarten, A., Madgar, J., Nissim-Rafinia, M., Yahav, Y, Goshen, R, et al., Am. J. Resir. Crit. Care Med., 155:1914-1920, (1997).

9. Mayelda, A., Helfman, D. M., Krainer, A. R., Mol Cell Biol., 13:2993-3001, (1993).

10. Nordqvist, K., Ohman, K., Akusjarvi, G., Mol. Cell Biol., 14:437-445, (1994).

11. Rve-Harel, N., Kerem, E., Nissim-Rafinia, M., Madjar, E., Goshen, R, Augarten, A., Rahat, A., et al., Am. J. Hum. Genet., 60:87-94, (1997).

12. Yang, X, Bani, M. R, Lu, S. J., Rowan, W., Ben-David, Y., Chabot, B., Proc. Natl. Acad Sci. USA, 91:6924-6928, (1994).

### BACKGROUND OF THE INVENTION

Pre-mRNA splicing is an essential process in the expression of most eukaryotic genes. The 5′ and 3′ splice sites, the pyrimidine tract and the branch site are the known cis elements that are necessary, albeit not sufficient, for the accuracy of splicing. Alternative splicing is a major mechanism for controlling gene expression. Flexibility in the recognition and the efficiency of alternative splice sites provide an avenue for regulating the process of alternative splicing. Recent studies have identified several factors essential for modulating alternative splicing in vitro and in vivo. These include several members of the SR proteins, including the SF2/ASF, the heterogeneous nuclear ribonucleoprotein A1 (hnRNP A1).

SF2/ASF has an essential role in regulating the use of proximal 5′ splice sites, promoting, both exon incusion and exon slipping in vivo and in vitro. The antagonistic splicing factor hnRNP A1 has an essential role in promoting the use of distal 5′ splice sites in vitro and in vivo. Increased levels of hnRNP A1 were shown to promote skipping of alternatively spliced exons in vitro. Such skipping depends on the context of the alternative exon as influenced by the size of the exon and by the relative strength of the polypyrimidine tract in the preceding intron. The effect of hnRNP A1 on exon skipping has not been studied in vivo.

Many mutations affecting the normal splicing pattern are known to cause a number of various human genetic diseases and pathological syndrome, among them cystic fibrosis.

Cystic fibrosis (CF) is a common severe autosomal recessive disease caused by mutations in the cystic fibrosis transmembrane conductance regulator (CFTR) gene. Several CFTR mutations affect the splicing of CFTR transcripts, leading to the generation of both correctly and aberrantly spliced transcripts. Among these are the 3849+10 kb C→T mutation and the mutation in the 5T allele. The 3849+10 kb C→T creates a partially active 5′ splice site in intron 19 which can lead to the insertion of a new 94 bp cryptic exon containing an in-frame stop codon between exon 19 and exon 20. The 5T allele can lead to high levels of transcripts lacking exon 9 which are translated into a non-functional CFTR protein.

The 3849+10 kb C→T mutation was found to be associated with a mild form of CF. Nevertheless, a marked variability in the disease severity is found among the patients. The 5T allele is the major mutation causing congenital bilateral absence of the vas deferens (CBAVD), In many of the men with CBAVD there are no other CF symptoms. The clinical status of individuals carrying the 5T allele can vary from healthy fertile males to severe CF. Furthermore, among different patients a wide variability in, the level of aberrantly spliced mRNA, transcribed from, these mutations, was found in the nasal epithelium and the epididymis. The variability in disease presentation was found to correlate with the level of aberrantly spliced RNA transcribed from these alleles. Variability in the levels of aberrantly spliced CFTR mRNA transcribed from the 3849+10 kB C→T mutation and the 5T allele was also found among different organs of the same individual. This variability also correlated with disease severity.

### SUMMARY OF THE INVENTION

The present invention is based on the finding that in vivo overexpression of the cellular human splicing factor hnRNP A1 activated exon slopping of CFTR alleles carrying different splicing mutations. One mutation, the 3849+10 kB C→T can lead to the inclusion of a cryptic exon and the other, the 5T, can lead to abnormal exon skipping. The invention is further based on the finding that overexpression of the viral specific factors E4-ORF3 and E4-ORF6 was able to modulate the splicing patterns towards normal patterns of the 5T and the 386 alleles, respectively.

Thus, die present invention concerns a method of treatment of an individual suffering from a disease resulting from abnormal expression of genes caused by aberrant splicing in cells, the method comprising:

administering to said cells of the individual, or to tissue or organs of said individual comprising said cells, an effective amount of an alternative splicing factor (ASF), whereby said abnormal expression shifts towards normal expression of the gene.

The term “treatment” in the context of the present invention does not necessarily mean complete curing of the disease, but may also refer to elevation of some of the undesired effects of the disease, or prevention of die most serious effects before they are manifested in the individual.

The term “a disease resulting from an abnormal expression of genes” refers to a genetic disease which may be caused by expression of non-normal (i.e. mutated) protein; a genetic disease caused by overexpression of a normal protein, or a genetic disease caused by under expression or lack of expression of a normal protein. The genetic disease which results from the abnormal expression of genes should be of the type that is caused by aberrant splicing of the gene, for example, exon skipping, or intron exclusion, which leads either to production of a mutated protein, overexpression of a protein, under expression or lack of expression of a protein, or combination of several of the above. The aberrant splicing may be in control regions or the coding regions of the cells.

The term “in cells” refers to the fact that the aberrant splicing manifested in specific types of cells in which the gene is expressed, should be expressed, or should have not been expressed.

An example of such genetic diseases is cystic fibrosis.

Examples of cells, tissue or organs comprising cells in which said aberrant splicing is manifested is: for cystic fibrosis: cells of the vas-deferens and the epididymis, cells of the lung and respiratory tissue, cells of the digestive tissue, nasal epithelium, etc.

The method of the invention concerns administering to said cells, or tissues or organs comprising said cells, as will be explained hereinbelow, an effective amount of an alternative splicing factor, (ASF) i.e. any factor which is known to modulate alternative splicing, for example, those mentioned in the publications referred to in the list of references, such as members of the SR protein family including the SF2/ASP and its antagonists, as well as the heterogenous nuclear ribonucleoprotein A1 (hnRNP A1).

The ASF may also be an agonist of the above naturally occurring factors, prepared by peptidomemetics, or by screening various libraries of compounds for the isolation, or the construction of an agent which is able to mimic the activity of naturally occurring ASFs.

Administration of said ASFs to the cells or tissues or organs comprising the cells of the individual, causes a shift in the expression of the gene responsible for the genetic disease towards normal expression. Where the abnormal expression is due to production of a mutated protein, said shift means that some level of a normal, non mutated protein is produced. Where said abnormal gene expression is caused by overexpression of a gene, said shift may be manifested by lowering the level of expression, and where said abnormal gene expression is caused by under expression or lack of expression of a gene, said shift may be manifested by increasing the level of expression of said protein.

In particular, where the genetic disease is cystic fibrosis, which is caused by the 3849+10 kb C→T mutation or a mutation in the 5T allele, the ASF may be, for example, the hnRNP A1, which can shift the splicing of the mutated gene, (which produced a mutated protein) so as to produce a higher level of correctly spliced product.

The ASF may be administered to the cells in any manner known in the art.

By one alternative, a nucleic acid sequence expressing the ASF may be inserted in an expression vector, such as a plasmid containing the coding region for the ASF under control of a suitable expression control element (such as a suitable promoter). Then, said cells of the individual are transfected with said expression vector in order to produce the ASF. Example of an hnRNP A1 expression plasmid is pCG-A1 (Krainer, A. R.) and an example of an expression vector containing the full length cDNA of adenovirus E4-ORF6 is pCMVE4-ORF6 (Nordqvist 1994). The expression vectors may be targeted to the desired cells or to the tissue or organ comprising the cells by any means known in the art for targeting compounds to specific tissue. For example, they may be administered directly to the cells. Where for example in cystic fibrosis the target cells or organs are die lungs, the expression vectors may be present within a carrier suitable for inhaling and penetrating the lungs.

By another alternative, the expression vector may be attached to targeting moiety, such as, for example, a suitable antibody or a ligand of a specific receptor which can specifically bind to the membranes of the desired cells and thus the expression vector to the desired cell population, or to the organ or tissue comprising said cell population. In such a case, the expression vector may be administered systemically, and the targeting moiety ensures that it reaches its proper target cell population.

By yet another alternative, the ASF may be administered as the protein product itself. For example, where the ASF is a protein, it may be administered as a protein, for example inside a suitable vehicle suitable for administration of proteins either by direct administration to the cells, tissue or organ as mentioned above (inhalation to lungs, injection to the organ, etc.), or alternatively by conjugating the vehicle, to a targeting moiety as described above. It should be noted that the ASF may be compounds other than proteins, which targeting moiety can then direct the product to the cells and may be any small molecule which can mimic the activity of naturally occurring ASF.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the minigene systems for the analysis of the 3849+10 kB C→T, 5T and 9T alleles. A: the 3849+10 kB C→T system, B: the 5T and 9T systems. Middle panel: the structure of the minigene, upper panel: aberrantly spliced transcripts, lower panel: correctly spliced transcripts. Boxes-exons of the CFTR gene, the number of each CFTR exon is marked within the box. The grey box indicate the cryptic 84 bp exon lines—CFTR introns. The splicing of the minigenes will retain CFTR intronic sequences at both ends of the transcript. The italic double lines indicate the junction between PCR fragments, which constructed the minigene. The numbers below the minigene structure indicate the bp length of the intronic and exonic sequences of each PCR fragment. The horizontal arrows mark the primers used for the RT-PCP The RT-PCR systems were designed to enable specific amplification of the minigene transcripts only. The forward primers are specific to CFTR intronic sequences, which are included in the minigene transcripts. The size of the RT-PCR products, from each splicing form, is indicated on the right end.

FIG. 2 shows the expression and splicing of p3849N and p3849M in COS-1 and HeLa cells. A: Southern analysis of the splicing pattern of RNA transcribed from p3849N and p3849M minigenes, using an exon 19 specific primer (C1-1D) as a probe. The number at the right end—size of the RT-PCR products. B: RT-PCR analysis of the splicing pattern of RNA transcribed from p3849N and p3849M. The amplification was performed with a fluorescent primer and analyzed using GENSCAN. The numbers above each profile indicate the size in base pairs.

FIG. 3 shows the effect of hnRNP A1 transient overexpresion, into COS-1 cells, on the splicing of p3849M pre-mRNA. A: RT-PCR analysis of the splicing pattern of RNA transcribed from p3849M. The amplification was performed with a fluorescent primer and analyzed using GENSCAN. The numbers above each profile indicate the size in base pairs. B: RT-PCR analysis of RNA transcribed from the endogenous and transfected hnRNP A1. The products were electrophoresed on an agarose gel. The RT-PCR system used for the analysis of the expression of hnRNP A1 (upper panel—transfected hnRNP A1, lower panel—endogenous hnRNP A1) is shown below. The size of each RT-PCR product is shown at the right end.

FIG. 4 shows the effect of hnRNP A1 transient overexpression into COS-1 cells, on the splicing of p5T and p9T pre-mRNA. RT-PCR analysis of the splicing pattern of RNA transcribed from p5T (upper panel) and p9T (lower panel). The amplification was performed with a fluorescent primer and analyzed using GENSCAN. The numbers above each profile indicate the size in base pairs.

FIG. 5 shows the effect of E4-ORF6 transient overexpression, into COS-1 and HeLa cells, on the splicing of p3849M pre-mRNA. A: RT-PCR analysis of the splicing pattern of RNA transcribed from p3849M. The amplification was performed with a fluorescent primer and analyzed using GENSCAN. The numbers above each profile indicate the size in base pairs. B: RT-PCR analysis of the RNA transcribed from the transfected E4-ORF6. The products were electrophoresed on an agarose gel. The RT-PCR system used for the analysis of the expression of E4-ORF6 is shown below. The size of the RT-PCR product is shown at the right end.

FIG. 6 is a schematic representation showing expression of correctly and aberrantly spliced transcripts of CFTR gene in the cell line.

FIG. 7 is a schematic representation showing the modulation of splicing as a result of overexpression of three different splicing factors, SRp20, SC35 and Htra2-β1 as compared to the untreated cell line 091398K.

### DETAILED DESCRIPTION OF THE INVENTION

I. Technical Procedures

I(a) Construction of the Minigenes

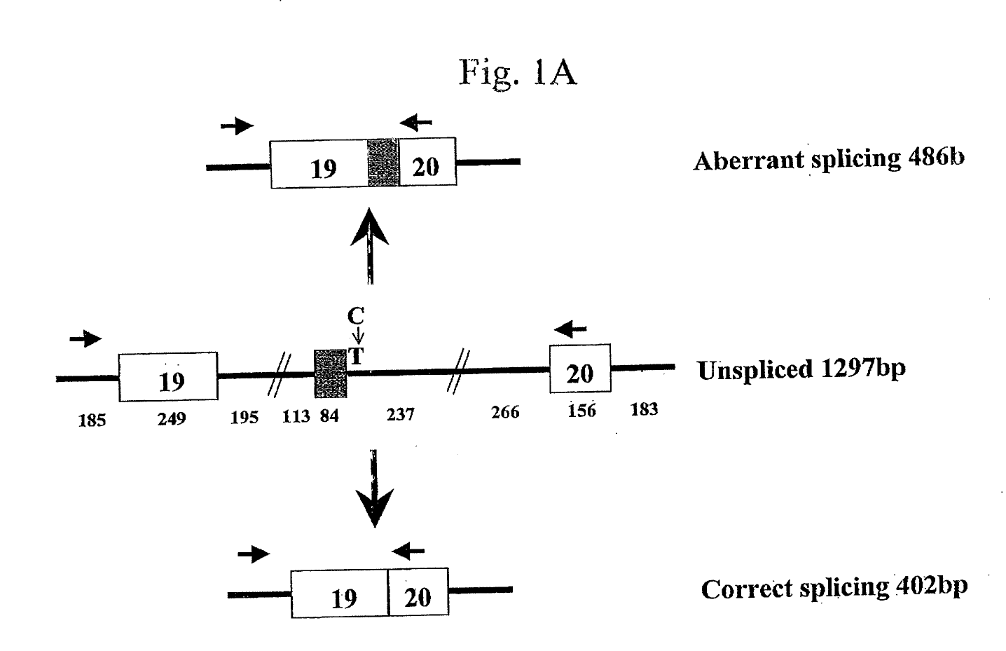

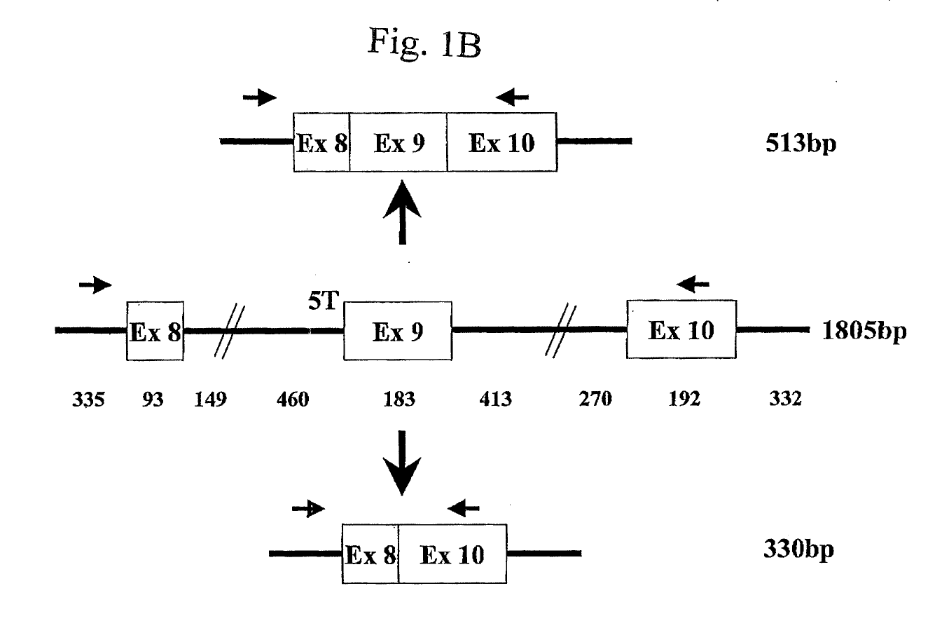

The minigene for the analysis of the 3849+10 kB C→T mutation (p3849M), and the control minigene with the normal sequence (p3849N) were constructed using genomic DNA from the same individual. This individual was a CF patient homozygous for the F508 mutation (in exon 10 of the CFTR gene). In the amplified region he had the normal sequence which is shown in FIG. 1a.

The minigenes (1668 bp) contained the following PCR fragments: exon 19 and part of its flanking introns (629 bp); a region from intron, 19 which included the alternative spliced cryptic 84 bp exon and its flanking sequence (434 bp); exon 20 and part of its flanking introns (605 bP) (FIG. 1a). The 3849+10 kB C→T mutation was introduced by site directed mutagenesis using Power-Cloning (see below). The same cloning approach was applied for the construction of the 5T (p5T, 2427 bp) and the 9T (p9T, 2431 bp) minigenes. These minigenes were constructed from the following PCR fragments: exons 8, 9 and 10 and part of their flanking introns (577 bp, 1054 bp and 794 bp, respectively) (shown in FIG. 1B). The PCR for the construction of p5T and p9T were performed on genomic DNA from the same individual (homozygous for the 5T allele), except for the fragment containing exon 9 of the p9T, which was amplified from a different individual who was homozygous for the 9T allele.

The minigenes were created by connecting the PCR fragments to each other and to the mammalian expression vector pSI (Promega) in a single step using Power-Cloning technology (patent pending, PCT IL 120339). This part of the work was performed at Gesher Advanced Biotecs. The pSI vector contains the SV40 promoter, enhancer and polyadenylation signal. Following the construction, the entire minigenes were sequenced, using an automated sequencer system (Applied Biotechnology). No sequence variations were identified between the minigenes and the genomic sequences, other than the mutations that had been deliberately introduced.

I(b) Plasmids of the Splicing Factors

The hnRNP A1 expression plasmid, pCG-A1 (Krainer, A. R.), contains the full-length human hnRNP A1 coding sequence (cDNA). The E4-ORF6 expression plasmid, pCMVE4-ORF6 (Nordqvist, 1994) contains the full length cDNA of adenovirus F4-ORF6 (Akusjarvi, G.).

I.(c) Cells and Transfections

COS-1 and HeLa cells were grown in DMEM with high glucose and MEM-E, respectively, supplemented with 10% fetal calf serum. The cells were grown in 10 cm tissue culture dishes, 10⁶ cells/dish were plated 24 hrs before transfection. Monolayer cells were transfected (or cotansfected with two different plasmids) using calcium phosphate and the 2xBBS co-precipitation technique. For the anlaysis of the 3849+10 kb C→T mutation, p3849M or p3849N were transfected. Cotransfections were performed with pCG-A1 or pCMVE4-ORF6. For the analysis of the 5T and 9T alleles, p5T or p9T were transfected. Cotransfections were performed with pCG-A1.

After the addition of the plasmids the cells were incubated for 24 hrs in 35° C. and 3% CO₂, followed by removal of the calcium phosphate precipitates the cells were further grown in the recommended medium (see above) for 48 hrs.

I.(d) RNA Preparation and Single-strand cDNA Synthesis

The transfected cells were harvested and lysed with ULTRASPEC RNA reagent, and total RNA was purified using the ULTRASPEC RNA Kit BIOTECX). cDNA was synthesized using 2.5 mM random hexamer mix (Pharmacia Fine Chemicals), 5 mM MgCl₂, 1 mM dNTP mix (Pharmacia), 100 units of Super-Script™ II Rnase H-Reverse (RT) (BRL), and 40 minutes of RNase inhibitor (Boehringer). The tubes were incubated at room temperature for 10 mins. at 42° C. for 40 mins. at 99° C. for 5 mins, and at 5° C. for 5 mins. Each cDNA synthesis experiment included a control sample in which all reagents except RNA were present.

I.(e) Analysis of the Splicing Pattern of the Minigene Transcripts

The cDNA of the different minigenes was amplified by PCR using recombinant Tag DNA polymerase (Boehringer). The primers used for the analysis of transcripts from p3849M and p3849N (shown in FIG. 1a):

19i5 5′ GCCCGACAAATAACCAAGTGA3′

specific for intron 19 of the CFTR gene and X20 5′ ATCCAGTTCTTCCCAAGAGGC 3′ specific for exon 20. X20 was fluorescently labeled with 6-FAM. The PCR products of the correctly and aberrantly spliced transcripts were 402 and 486 bp, respectively.

The primers used for the analysis of the poly T minigenes (p5T and p9T) were:

8Ri5 5′ TGCATTAATGCTATTCTGATTC 3′

specific for intron 8 of the CFTR gene and F10Rx3 5′ TTGGCATGCTTTGATGACGC 3′ specific for exon 10 (shown in FIG. 1b). F10Rx3 was fluorescently labeled with 6-FAM. The PCR products of the correctly and aberrantly spliced transcripts were 513 and 330 bp, respectively.

RNA-less reactions were used as controls. The cDNA samples were heated at 94° C. for 3 mins. and then subjected to 35 cycles of: 94° C. for 1 min., 55° C. for 30 seconds, and 65° C. for 1 min., followed by final extension for 7 mins. at 65° C. The PCR was performed under semi-quantitative conditions as determined by serial tertiary dilution prior to the experiments (data not shown). The amount of PCR product required to give an appropriate fluorescent signal was empirically determined by analysis of serial diluted PCR products on polyacrylamide gels. The suitable product amounts were 1-2% of the total PCR products. The analysis was performed as previously described, in brief: 11 of each PCR product was mixed with 0.41 of a TAMRA-labeled commercial size standard (Genescan 500-Tamra, Applied Biosystems) and run on an ABI 377 system. The analysis was performed using GENSCAN software (version 2.X). The level of the aberrantly or correctly spliced transcripts was determined as: (the peak area of the signal of the aberrantly or correctly spliced PCR product)/(the peak area of the signal of the aberrantly spliced PCR product+the peak area of the signal of the correctly spliced PCR product).

I.(f) Expression of the Splicing Factors

To verify the expression of the splicing factors, a PCR reaction was designed to amplify each of the cDNAs of the splicing factors. The primers used for hnRNP A1 analysis were:

pCG 5′UTR: GACGCCATCCACGCTGTT, which is specific for the 5′ untranslated region (UTR) derived from the pCG vector, and

A1exp-5′: AAAGTCTCTTCACCCTGC, which is specific for the 5′ UTR of the endogenous hnRNP A1 gene; both of these were used as forward primers, and

A1exp-3′: AAGTGGGCACCTGGTCTTTG was used as a reverse primer. All three hnRNP A1 primers were present in the same reaction. The primers used for E4-ORF6 analysis were:

ORF6exp-5′: CCCGAATGTAACACTTTGAC as a forward primer, and

ORF6exp-3′: CGGTACCATATAAACCTCTG as a reverse primer.

For both reactions the cDNA samples were heated at 94° C. for 3 mins., and then subjected to 30 cycles of: 94° C. for 1 mins., 52° C. for 30 seconds, and 65° C. for 1 min., followed by final extension for 7 mins. at 65° C.

RESULTS

### EXAMPLE 1

### Splicing of the 3849+10 kb C→T Minigenes in vitro

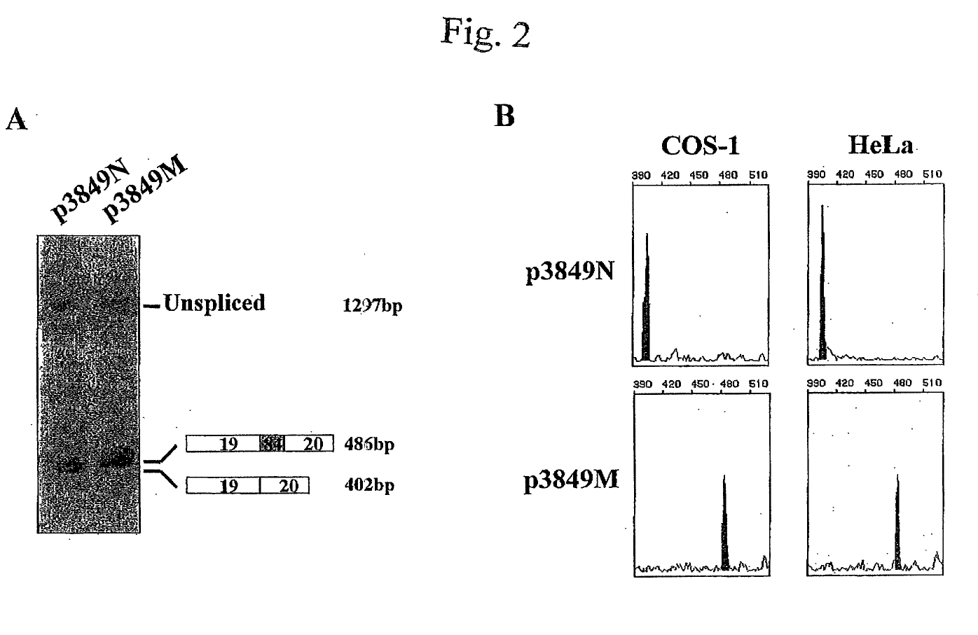

CFTR containing genomic sequences from exon 19, the cryptic 84 bp exon, exon 20 and their upstream and downstream flanking sequences were introduced into the pSI expression vector (FIG. 1a). The minigenes containing the 3849+10 kb C→T mutation (p3849M) or the normal sequence p3949N) were transfected into HeLa and COS-1 cells. Both minigenes were successfully expressed and spliced in these cells (FIG. 2 and Table 1). All the spliced transcripts from p3849M included the cryptic “84 bp exon” (486 bp RT-PCR product in FIG. 2). No correctly spliced transcripts were detected from this minigene. All the transcripts from p3949M were correctly spliced (402 bp RT-PCR product in FIG. 2), thus, the 84 bp in this minigene were not recognized as an exon.

No differences in splicing pattern were observed upon transfection with different amounts of p3949M or p3849N DNA. However, the amounts of spliced transcripts were higher upon transfection with 5 μg of p3849M or p3849N than upon transfection with 1 or 2.5 μg. No further increase in the intensity was found upon transfections with 10 or 20 μg. Thus, in the subsequent cotransfection experiments 5 μg of the minigenes were used.

### EXAMPLE 2

### The Effect of Overexpression of the Cellular hnRNP A1 on the Splicing of the 3849+10 kb C→T Minigenes

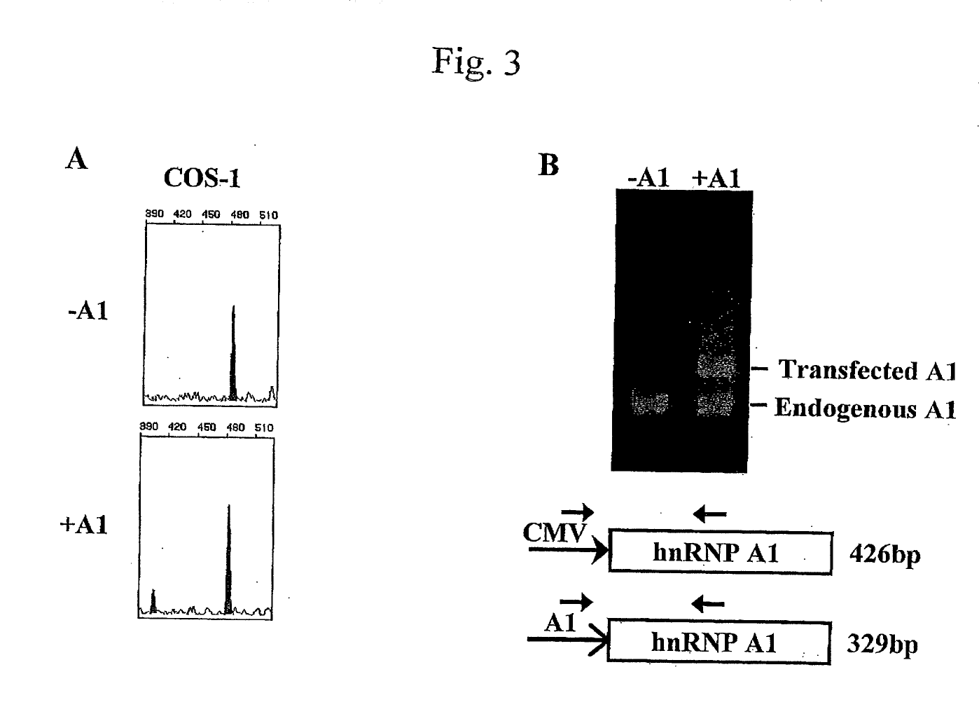

Transient cotransfection into COS-1 cells, of the 3849+10 kb C→T minigene, p3849M, and a human hnRNP A1 cDNa known to promote exon skipping, pCG- A1 (5 or 10 μg), resulted in the generation of normal spliced transcripts (FIG. 3a). In repeated experiments, overexpression of hnRNP A1, led to correct splicing of 12% of the total minigene RNA transcripts (FIG. 3a and Table 1). Increasing the amount of pCG-A1 DNA did not increase the proportion of correctly spliced RNA. Cotransfection with only 2.5 μg pCG-A1 did not permit any correct splicing. Transient cotransfection of p3849N and 5 μg pCG-A1, as expected, did ot affect the splicing pattern of the p3849N minigene. In each experiment the expression of pCG-A1 was verified by RT-PCR analysis (FIG. 3b). Transient cotransfection into HeLa cells of p3849M and pCG-A1 did not affect the splicing pattern of RNA transcribed from the minigene (data not shown). However, RT-PCR analysis of hnRNP A1 revealed that pCG-A1 was not expressed in the HeLa cells, which might account for the unaffected splicing pattern.

### EXAMPLE 3

### The Effect of Overexpression of the Cellular hnRNP A1 on the Splicing of the PolyT Minigenes

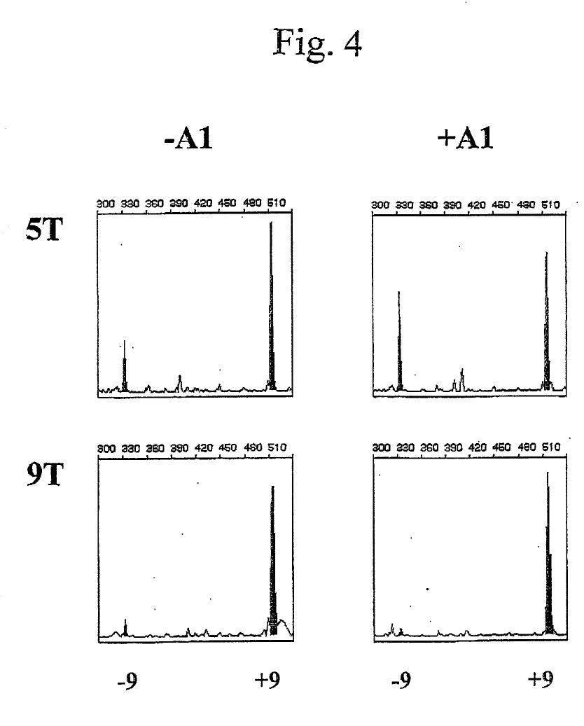

CFTR genomic sequences from exons 8, 9 and 10 and their flanking sequences were inserted into the pSI expression vector (FIG. 1b). 5 μg of the minigene containing the 5T (p5T) or the 9T (p9T) allele were transfected into COS-1 cells. As can be seen in FIG. 4, both minigenes were successfully expressed and spliced.

Transfection of p5T generated two splicing products: 24% of the transcripts were aberrantly spliced (330 bp) and the rest (76%) were correctly spliced (513 bp) (FIG. 4 and Table 2). Upon transfection of p9T only 3% of the spliced RNA was aberrantly spliced (FIG. 4 and Table 2).

Transient cotransfection into COS-1 cells, of p5T and pCG-A1 (5 or 10 μg), resulted in a substantial increase in aberrantly spliced transcripts (44%) (FIG. 4 and Table 2). Transient cotransfection of p9T and pCG-A1 (5 μg) did not affect the p9T minigene splicing pattern.

### EXAMPLE 4

### The Effect of the Overexpression of the Viral E4-ORF6 on the Splicing of the 3849+10 kb C→T Minigenes

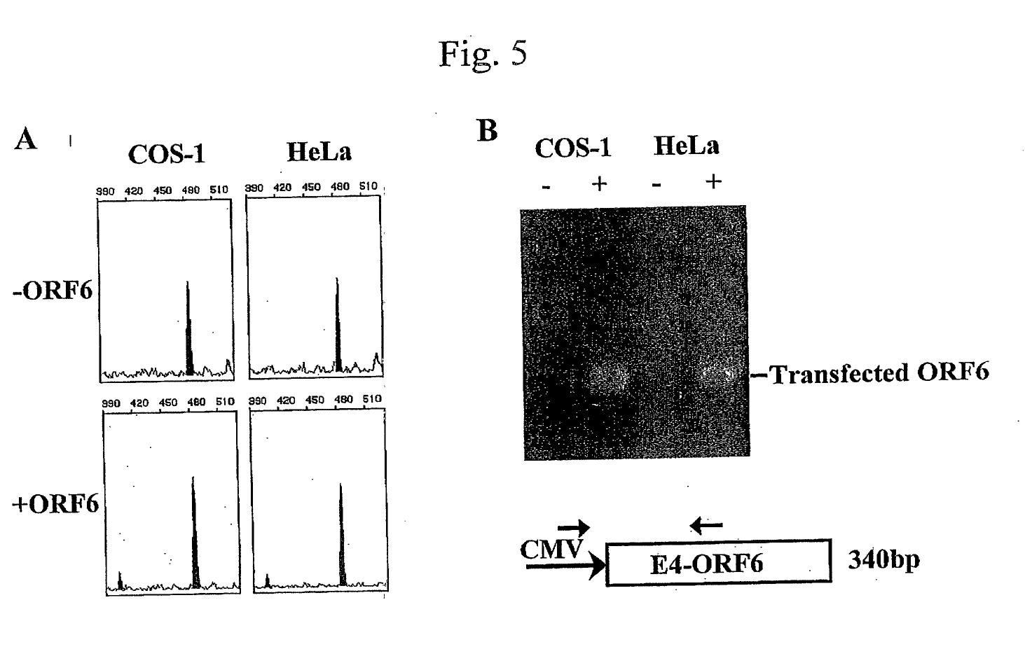

It has been hypothesized that upon infection by adenovirus, and expression of its splicing factors, the cellular splicing activity might be affected and thus the splicing pattern of CFTR transcripts carrying splicing mutations might be modified. In order to test this hypothesis the effect of one of the adenoviral splicing factors, E4-ORF6, was known to have a similar activity to hnRNP A1. Transient cotransfection into both COS-1 and HeLa cells of p3849M and the adenovirus E4-ORF6 cDNA (pCMVE4-ORF6) (5 or 10 μg) generated correctly spliced transcripts (FIG. 5). In repeated experiments 9% of total p3849M RNA in COS-1 cells, no effect on the splicing pattern of the minigene was found, as expected for a minigene with the normal sequence. In each experiment the expression of transfected pCMVE4-ORF6 was verified by RT-PCR analysis (FIG. 5b).

### EXAMPLE 5

### Splicing Factors that Regulate Alternative Splicing in vivo

Pre-mRNA splicing is an essential process in the regulation of expression of most eukaryotic protein-coding genes. The 5′ and 3′ splice sites and the branch site, which exhibit limited sequence conservation, are necessary but not sufficient for the accuracy of splicing. In the case of constitutively spliced pre-mRNA strict fidelity is necessary for correct protein synthesis. However, many cellular and viral genes are regulated by alternative splicing. Several factors are essential for modulating constitutive as well as alternative splicing in vitro and in vivo. This includes cellular proteins from the SR and the hnRNP families (such as ASF/SF2, SRp20, SRp35, PTB, hnRNP A1, etc.), and the non essential SR-like splicing factor Htra2-β1, known to promote exon inclusion and/or skipping of endogenous genes programmed to undergo alternative splicing. In addition, there are cellular factors that regulate the activity of SR splicing factors (such as p38 kinase, kinases from the Clk family and the SR protein inhibitor p32). This regulation involves phosphorylation and compartmentalization of the SR splicing factors.

Establishment of ex vivo cellular systems carrying the 3849+10 kb C→T and the 5T mutations

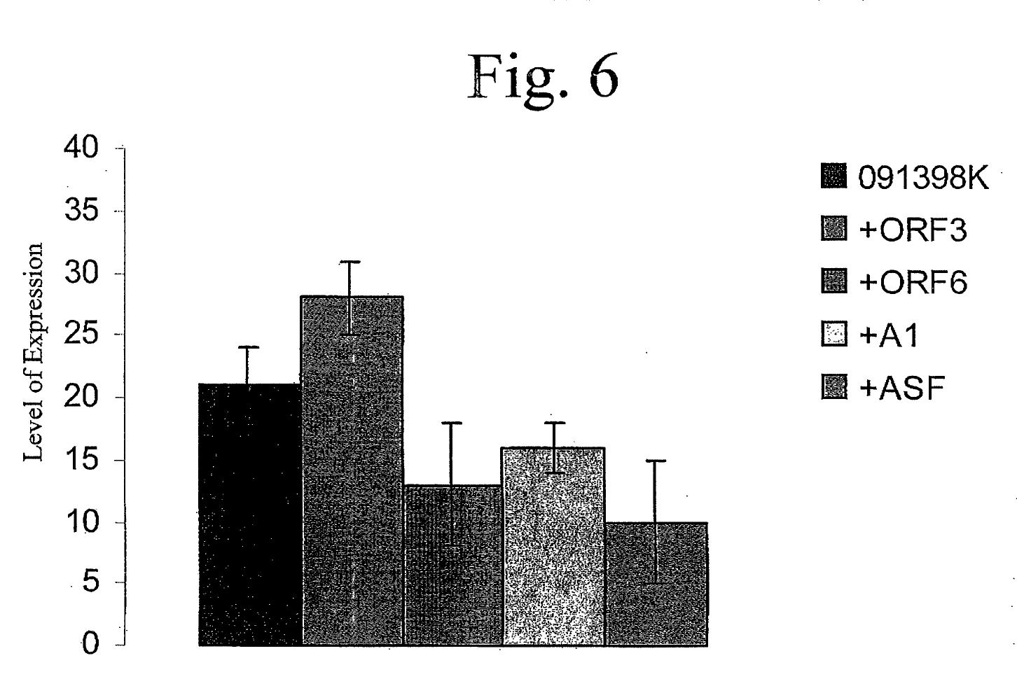

A polyp sample from a CF patient, compound heterozygous for the splicing mutation 3849+10 kb C→T and the W 1282X mutations was obtained. This patient suffered from nasal polyps and underwent nasal polypectomy. An epithelial cell line (091398K) was established from the polyp sample in collaboration with Dr. J. Yankaskas from the University of North Carolina. Preliminary analysis showed that the CFTR gene is expressed in the cell line and both correctly and aberrantly spliced transcripts are generated (FIG. 6). Thus a system in which the effect of splicing factors or) a native CFTR gene could be studied, was established.

Studying the modulation of the splicing pattern by a series of cellular and viral splicing factors

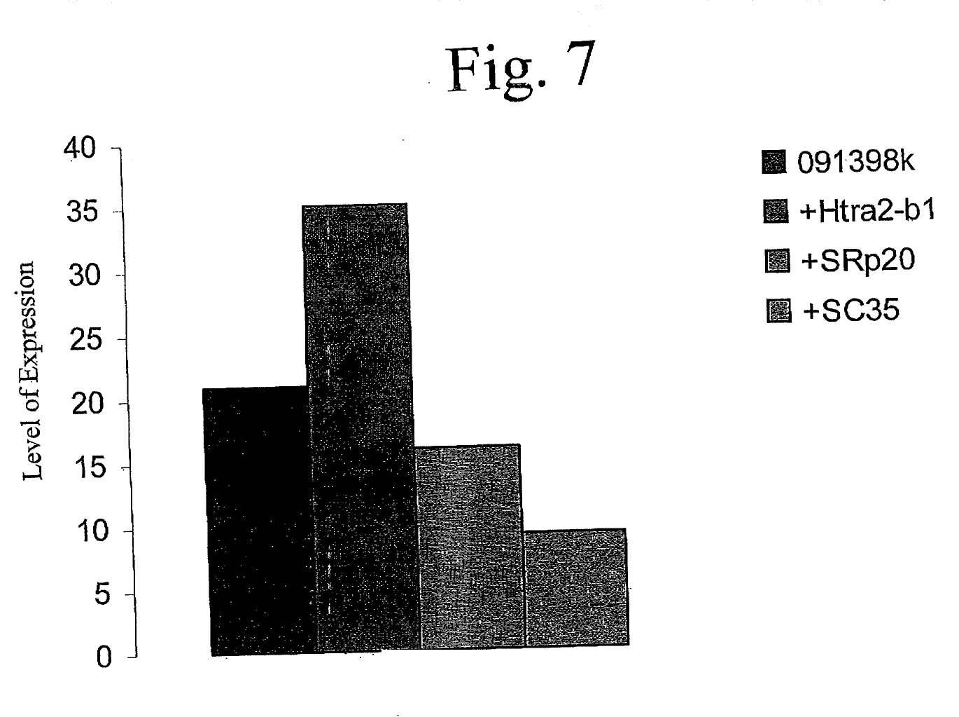

Transient transfections of 091398 k cells with cellular factors were performed, using DAC-30. The use of DAC-30 resulted in transfection efficiency of ˜60%. In these experiments splicing factors were analyzed that were shown to affect the splicing pattern of minigene carrying the splicing mutations: ASF/SF2, hnRNPA1, E4-ORF3 and E4-ORF6. The results showed that all these factors modulated the splicing pattern of CFTR transcripts (FIG. 6). The ASF/SF2, hnRNP A1 and E4ORF6 promoted exon skipping and led to a decrease in the level of aberrantly spliced transcripts (FIG. 7). The most significant effect was achieved with ASF/SF2 which led to a decrease of the aberrantly spliced transcripts from 21% to 11%. The viral factor E4-oRF3 slightly promoted exon inclusion and led to an increase in, the level of aberrantly spliced transcripts (21% to 28%). Thus, the mean effect could have reached ˜35%.

Since only ˜60% of the cells were transfected, the results indicate that in the transfected cells, the mean effect could have reached a complete abolishing of the aberrantly spliced transcripts. These results suggest that the 091398 k cell line might be suitable for function CFTR analysis.

This analysis was extended by studying three additional cellular splicing factors: two proteins from the SR family, SRp20, Sc35 and the non-essential SR-like protein Htra2-β1. Overexpression of these factors in the 091398 k cells resulted in the modulation of the splicing pattern (FIG. 7). The SR proteins SC35 and SRp20, promoted exon skipping and led to a decrease in the level of the aberrantly spliced transcripts to 9% and 16% respectively. This effect is more significant from that of ASF/SF2 and hnRNPA1 that were used in previous studies.

The non-essential SR-like protein Htra2-β1 promoted exon inclusion and led to an increase in the level of the aberrantly spliced CFTR transcripts to 35%. This effect is similar to that of the viral splicing factor ORF3.

It is important to note that the splicing factor Htra-β1 was recently used to modulate the splicing pattern in another inherited disease, spinal muscular atrophy (SMA (Yvonne Hofmann et al., PNAS, 97(17):9618-9623, Aug. 15, 2000)). In this disease two almost identical copies of the SMN genes are involved. In most patients two copies of the SMN1 gene are disrupted, and the disease severity is correlated with the level of inclusion of exon 7 of the SMN2 gene. Overexpression of the splicing factor Htra2-β1 promoted significantly the inclusion of exon 7 in SMN2 minigenes to 90%.

In summary, the repertoire of factors that can modulate the splicing pattern of CFTR alleles carrying splicing mutations was extended. Two additional factors which promote a decrease in the level of aberrantly spliced transcripts carrying the 3849+10 kb C→T mutation were identified.

1. A method of treatment of an individual suffering from a disease resulting from an abnormal expression of genes caused by aberrant splicing in cells, the method comprising:
Administering to said cells of the individual or to tissue or organs of said individual comprising said cells, an effective amount of an alternative splicing factor (ASF), whereby said abnormal expression shifts towards normal expression of the gene.

2. A method according to claim l, wherein said disease is cystic fibrosis.

3. A method according to claim 2, wherein the aberrant splicing is caused by a mutation 3849+10 kb C→T.

4. A method according to claim 2, wherein the aberrant splicing is caused by a mutation in the 5T allele.

5. A method according to claim 1, wherein the ASF is selected from the group consisting of:
(i) a member of the SR protein;
(ii) heterogeneous nuclear ribonucleoprotein A1;
(iii) viral factor E4-ORF3;
(iv) viral factor E4-ORF6; and
(v) an agonist of any one of (i) to (iv).

6. A method according to claim 1, wherein the ASF is administered to the cells or to the tissue or organs comprising the cells in a pharmaceutically acceptable vehicle.

7. A method according to claim 6, wherein the ASF is administered directly to the cells or to the tissue or organ comprising the cells.

8. A method according to claim 6, wherein the ASF is attached to a targeting moiety capable of binding specifically to said cells.
